(19) United States
(12) Patent Application Publication
(10) Pub. No.: US 2015/0126893 A1
(43) Pub. Date: May 7, 2015

(54) Method For Determining If Surgically-Implanted Hardware Should Be Removed

(71) Applicants: Maryrose Cusimano Reaston, Tulsa, OK (US); Frank J. Tomecek, JR., Tulsa, OK (US); Phil Reaston, Tulsa, OK (US)

(72) Inventors: Maryrose Cusimano Reaston, Tulsa, OK (US); Frank J. Tomecek, JR., Tulsa, OK (US); Phil Reaston, Tulsa, OK (US)

(21) Appl. No.: 14/069,728

(22) Filed: Nov. 1, 2013

(51) Int. Cl. A61B 5/00; A61B 5/05; A61B 5/0488

(52) U.S. Cl. CPC A61B 5/4851; A61B 5/0488; A61B 5/05
USPC 600/546; 600/594

(57) ABSTRACT

A method for determining if surgically-implanted cervical, thoracic or lumbar hardware should be removed from an individual. The method utilizes diagnostic data acquired from a test protocol performed by an electrodiagnostics functional assessment (EFA) device. The diagnostic data is selected from the group consisting of electromyography (EMG), range-of-motion (ROM), functional capacity assessment (ECA), muscle data, myofascial data, structural data, nerve data or ischemic data.

### TECHNICAL FIELD

The invention generally pertains to surgically-implanted hardware, and more particularly to a method for determining if surgically-implanted cervical, thoracic or lumbar hardware should be removed.

### BACKGROUND ART

Stabilization and fusion of a person's lumbar spine may be performed by using various anterior and posterior surgical techniques and a wide range of hardware, including screws, spinal wires, and vertebral cages. The hardware is designed to stabilize the spine after fusion. Post operative imagining is typically used to determine the progress and positioning of the hardware. The type of imaging depends on the type of hardware. Currently, there is no reference standard for non-invasive imaging evaluation of fusion.

Radiography is the noninvasive modality most commonly used for the assessment of fusion. Radiology can have limited benefits, especially if breakage of the hardware or incorrect placement of the hardware is suspected. Flexion and extension views have been recommended for routine assessment of hardware post fusion, but there is no objective clinical data to support this use for the routine assessment of fusion.

A computer tomography (CT) scan is another modality for imaging bony detail in a person's spine during post surgical evaluation. However, the quality of CT images may be degraded because of artifacts due to metallic implants. While magnetic resonance imaging (MRI) also has artifacts, the artifacts seen on CT are not limited to the area immediately adjacent to the surgical area. Also with both CT and MRI, movement artifact(s) also significantly affect evaluation of most surgical changes.

MRI is one of the better imaging tools to evaluate postoperative changes in the spine, but it is much more sensitive to detect soft tissue and neuro-vascular changes than it is in detecting bone and arthrodesis. It is particularly useful for detecting and monitoring infection or postoperative scar. However, magnetic susceptibility artifact(s) may be a problem, particularly in the presence of stainless steel devices. Recent hardware is made of titanium alloys and even though they produce less severe magnetic artifacts, the artifacts still present a significant obstacle to visualization of areas in close proximity to the hardware.

In order for any kind of imaging to be beneficial, pre and post-operative comparisons are necessary. Other techniques such as ultrasonography, bone scans, and myelography have little or no benefit in assessing post-operative hardware.

The primary types of hardware most commonly used in spinal fusion are: interbody spacers, plates or rods with pedicle screws and translaminar or facet screws. Unfortunately, the hardware may need to be removed because of infection, damage or pain. A significant issue facing surgeons is trying to distinguish patients who have hardware failures, loosening or pseudoarthrosis, from patients who have pain due to hardware irritating the paraspinal soft tissue. Currently, a method of assessing if hardware removal will be beneficial is the injection of a local anesthetic around the site of the pain. The patient is asked to show their doctor the point of tenderness, an injection (typically lidocaine) is injected at the site of tenderness and then the patient is asked if the pain is better. If they report a decrease in pain then if might be that the hardware is contributing to the pain. This is the current method is use to determine if hardware removal will be beneficial. This method is not objective, fraught with subjectivity and does not confirm to the guidelines of evidence based medicine. This is not only problematic but may subject the patent to unnecessary surgery to remove the hardware.

Hardware removal is the most common elective orthopedic procedure, accounting for 30-50% of all planned orthopedic surgical procedures. The surgeries not only require a second surgical procedure in tissue that is already scarred but also poses a risk for nerve injury and recurrent spinal deformity or delayed fusion failures. It is essential that surgeons recognize lumbar hardware removal as a major surgery. Most series report a complication rate ranging from 3% to 20%. Complications include infection and bleeding, which is sometimes a challenge to control due to large bony defects after pedicle screw removal. Additionally, hardware removal may alleviate but not fully correct the problem; and retained hardware may be only one of several pain generators in the post-operative spine. Pain is the most predominant indication for removal of the hardware. Doctors have even named this condition “painful hardware syndrome”, yet there is no diagnostic code that reflects such as a syndrome. As a result, there is an overwhelming need for an objective method to evaluate if hardware needs to be removed after surgery.

A case study from the Texas Scottish Rite Hospital for Children reviewed results after implanted hardware removal in scoliosis patients. Unfortunately, 8% of the patients who underwent hardware removal required reoperation due to pain; and 5% of the patients required reoperation due to infection. Overall, after the removal of the hardware, there was an immediate progression of 3% in the thoracic and 6% in the lumbar scoliotic curves and sagittal plane progression including kyphosis. Instead of alleviating the problem, the removal of the hardware worsened the patient's curvature and sagittal balance. Therefore, it is essential, especially in long fusions for scoliosis, that the physician and patient are fully confident that the patient will be an appropriate candidate hardware removal. In the case of scoliosis patients, even those with a solid fusion, hardware removal may not be in their best interest; the potential benefit should outweigh the risks to the patient in this type of procedure.

A search of the prior art did not disclose any literature or patents that read directly on the claims of the instant invention. However, the following U.S. patents are considered related:

| Pat. No. | INVENTOR | ISSUED |
|---|---|---|
| 4,887,595 | Helnig | 19 Dec. 1989 |
| 5,055,104 | Ray | 8 Oct. 1991 |

The U.S. Pat. No. 4,887,595 discloses a surgically implantable device for maintaining the relative positions of spinal bodies of a spinal column. The device comprises a plate for connection with a first spinal body. The plate portion has a first side surface for facing the first spinal body and a second side surface. The plate portion has an opening which extends through the first and second side surfaces for receiving a fastener to connect the plate portion with the first spinal body.

The U.S. Pat. No. 5,055,104 discloses a fusion cage that is utilized when an intervertebral fusion is requested. The fusion cage preserve the disc space and the implanted by an anterior material to the lower back. The cage can be removed after the vertebrae have become fused together and without disrupting that fusion, thus guarding against possible rejection of the cages by the patient's body.

For background purposes and indicative of the art to which the invention relates, reference may be made to the following remaining patents found in the patent search.

| Pat. No. | INVENTOR | ISSUED |
|---|---|---|
| 4,157,085 | Austad | 5 Jun. 1979 |
| 5,284,130 | Ratliff | 8 Feb. 1994 |
| 5,607,480 | Beaty | 4 Mar. 1997 |
| 5,816,811 | Beaty | 6 Oct. 1998 |
| 5,954,638 | Spranza, III | 21 Sep. 1999 |
| 8,409,075 | Chu | 2 Apr. 2013 |

### DISCLOSURE OF THE INVENTION

In one of the most effective embodiments, the method for determining if surgically-implanted hardware should be removed from an individual utilizes diagnostic data acquired from a test protocol performed by an electrodiagnostic functional assessment (EFA) device. The EFA functions in combination with a computer operating proprietary software for medical evaluation. The EFA test protocols comprise electromyography (EMG), range-of-motion (ROM) and functional capacity assessment (FCA), along with muscle evaluation, to acquire any analyze specific data that will disclose if hardware removal, typically after cervical or lumbar surgery, is necessary. The EFA diagnostic data allows a healthcare professional to receive specific information on muscles, range of motion, functional capacity assessment, compliance, nerve injury and/or ischemic changes which will provide an objective indication that hardware removal is necessary.

The actual steps that are performed in the method are:
a) select an individual who is a candidate for either pre-surgical hardware implantation, post-surgical hardware implantation or pre-removal of surgically-implanted hardware,
b) determine if the hardware removal has organic or objective etiology,
c) prepare said EFA device for selected test protocol,
d) perform at least one rest and return to rest test protocol for EMG, if selected,
e) perform at least one rest and return to rest test for ROM, if selected,
f) perform at least one rest and return to rest test for FCA, if selected,
g) acquire test protocol results and diagnostic data, wherein the diagnosis data will disclose if muscle, ischemic or nerve damage is present in the area of the surgically-implanted hardware, and
h) use the diagnostic data to determine whether pre-implanted hardware should be implanted, or if post-implanted hardware should be removed.

In view of the above disclosure, the primary object of the invention is to provide a method for healthcare professionals to use a medical diagnostic unit (EFA) to acquire EMG, ROM and FCA data that will provide a specified objective determination if hardware removal post cervical, thoracic or lumbar surgery is needed.

In addition to the primary object of the invention, it is also an object of the invention to provide a method for determining if surgically-implanted hardware should be removed that:
can quickly and accurately provide a determination if hardware should be removed,
utilizes specific test protocols,
utilizes steps that are easy to administer,
can be performed by a single medical professional,
utilizes multiple test protocols to ensure the highest degree of accuracy,
utilizes an EFA device that is easy to transport to any location,
can be used in combination with other modalities such as X-ray, CT and/or MRI,
can be effectively used pre-hardware implantation, post-hardware implantation or pre-hardware removal, and
is cost effective from both a healthcare provider and patient's point of view.

These and other objects and advantages of the present invention will become apparent from the sub sequent detailed description of the preferred embodiment and the appended claims taken in conjunction with the accompanying drawings.

### BRIEF DESCRIPTION OF THE DRAWINGS

FIG. 1 is a flow diagram showing each of the steps that are utilized in the test protocol.

FIG. 2 is an elevational view showing an individual undergoing a test protocol from an EFA device. Multiple wireless leads are attached to the individual's back adjacent the site of hardware implantation. Diagnostic data is wirelessly sent from the leads to the EPA for diagnosis.

### BEST MODE FOR CARRYING OUT THE INVENTION

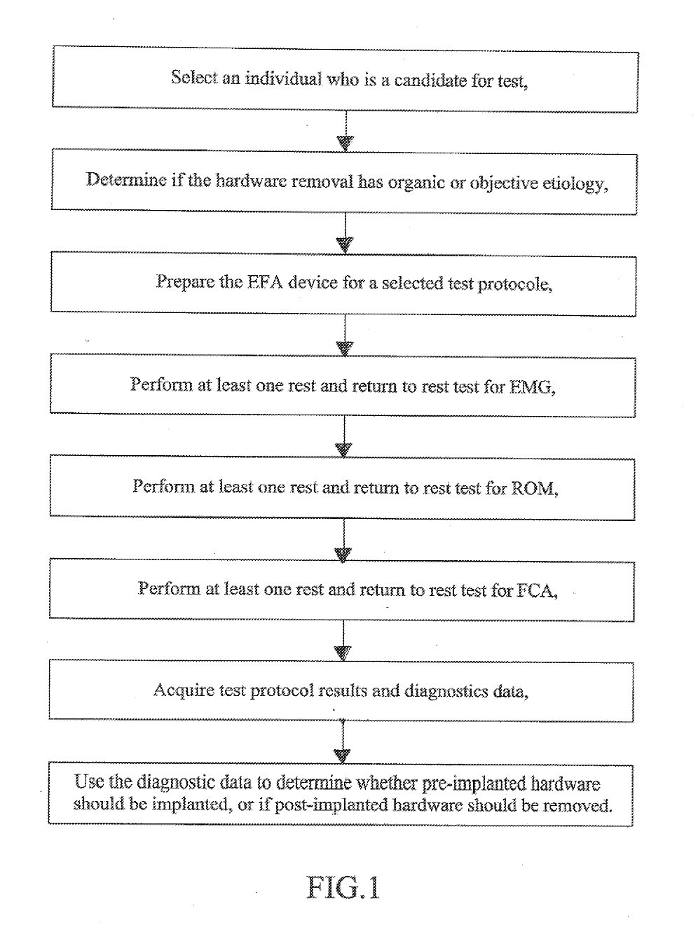

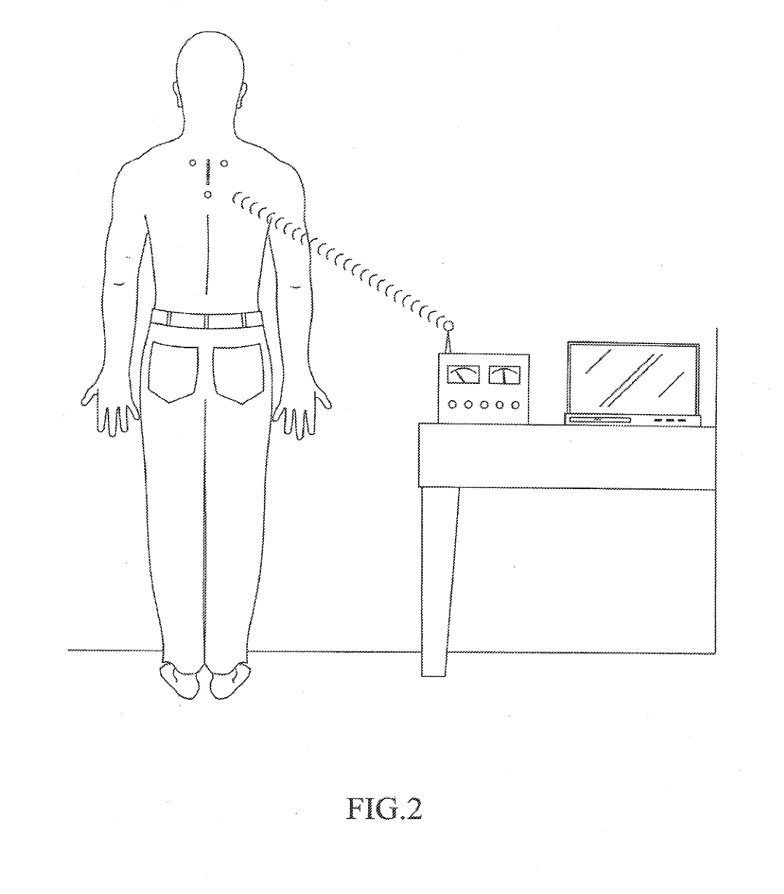

The best mode for carrying out the invention is presented in terms that disclose a method for determining if surgically-implanted hardware should be removed from a person. The method as disclosed in steps, described and shown in FIGS. 1-2, provides an effective means for healthcare professionals to determine if surgically-implanted hardware must be removed, should be removed or should remain implanted. The method is accomplished by analyzing diagnostic data acquired from a test protocol from an Electrodiagnostic Functional Assessment device (EFA). There are currently two main versions of the EFA: the EFA-2 which utilizes wired technology, and the EFA-3 which utilized wireless technology. Both the EFA-2 and the EFA-3 are issued U.S. patents to the applicant and are incorporated herein by reference (EFA-2 U.S. Pat. No. 8,535,224 and EFA-3 U.S. Pat. No. 8,568,312).

Regardless of which version is employed, the EFA is utilized to acquire and provide the diagnostic data that is based on the testing protocol to determine if surgically-implanted hardware should be removed. The diagnostic data discloses the information that indicates whether hardware removal is appropriate. The method is conducted in the event there is no infection, or broken or dislodged hardware in the person, of if there are no other objective findings disclosed from other diagnostic tests, such as x-rays, CT scans or MRI.

The EFA simultaneously monitors electromyography, range-of-motion and functional capacity assessment. The muscle groups monitored for the instant method are paraspinal, cervical, quadratus lumborum, gluteal, upper and lower extremities and muscles in the sacro iliac region. The EFA functions in tandem with a computer having a touch screen or keyboard and running proprietary software which correlates muscle activity, ischemic activity with the electromyography, range-of-motion, and a functional capacity assessment.

The initial process for utilizing the instant method 10 is to perform a pre-surgical baseline EPA test protocol. A post surgical evaluation is then done after 3-months post surgery for comparison to the pre-surgical baseline and as a reference should there be a potential hardware problem(s). The EFA test protocol is then repeated and compared to the prior baseline test protocol. In the event of potential hardware problem(s) a pre-surgical and post surgical after 3-month baseline test protocol is not required. The method does require an EPA assessment at the time of the consult for potential hardware removal. The method will assess if there is a problem(s) resulting from failed surgery, a hardware problem or a myofascial problem, and be able to identify the issue and prevent unnecessary surgery.

The test protocols for the method include but are not limited to rest and return to rest readings, ROM readings and FCA for the specific body parts evaluated.
a) select an individual who is a candidate for either pre-surgical hardware implantation, post-surgical hardware implantation or pre-removal of surgically-implanted hardware,
b) determine if the hardware removal has organic or objective etiology,
c) prepare said EFA device for selected test protocol,
d) perform at least one rest and return to rest test protocol for EMG, if selected,
e) perform at least one rest and return to rest test for ROM, if selected,
f) perform at least one rest and return to rest test for FCA, if selected,
g) acquire test protocol results and diagnostic data, wherein the diagnosis data will disclose if muscle, ischemic or nerve damage is present in the area of the surgically-implanted hardware, and
h) use the diagnostic data to determine whether pre-implanted hardware should be implanted, or if post-implanted hardware should be removed.

Data will not only yield specific information about muscles, nerves, spinal changes and ischemic changes, but will yield information about effort which can be an indication of symptom magnification and could be an indicator that hardware removal is not necessary. The test protocol for the method also includes data from the paraspinal muscles and surrounding musculature to identify if the data correlates to patient complaints and hardware location. The test protocol is performed bilaterally as often a hardware problem(s) will be present unilaterally. The method indicates if the diagnostic data discloses no ischemic changes or nerve changes in the paraspinal muscle which indicates there is no need for hardware removal. If the surrounding musculature has changes not consistent with complaints, the method also indicates that hardware removal is not required. The method does indicate if muscle, ischemic and or nerve data is present in the area of complaint, and this a good indication that hardware removal is required. The method also utilizes the EMG, ROM and FCA data in multiple related combinations with flexion, extension and rotation; lifting, pulling and pushing; and spinal range of motion.

While the invention has been described in detail and pictorially shown in the accompanying drawings it is not to be limited to such details, since many changes and modification may be made to the invention without departing from, the spirit and the scope thereof. Hence, it is described to cover any and all modifications and forms which may come within the language and scope of the claims.

1. A method for determining if surgically-implanted hardware should be removed from an individual, wherein said method utilizes diagnostic data acquired from an Electrodiagnostic Functional Assessment (EFA) device.

2. The method as specified in claim 1 wherein said EFA device's diagnostic data is acquired by testing means selected from the group consisting of electromyography (EMG), range-of-motion (ROM) and functional capacity assessment (FCA).

3. The method as specified in claim 1 wherein said EFA further monitors muscles that are selected from the group consisting of paraspinal, muscles, cervical muscles, upper extremity muscles, lower extremity muscles, gluteal muscles, hip muscles and sacro iliac muscles, in combination with the EMG, ROM and FCA.

4. The method as specified in claim 1 wherein said EPA device further monitors medical data that is selected from the group consisting of myofascial data, structural data, nerve data and ischemic data.

5. The method as specified in claim 1 wherein said EMG data is evaluated bilaterally with spinal range of motion data.

6. The method as specified in claim 1 wherein said EMG data is evaluated bilaterally with FCA data.

7. The method as specified in claim 1 wherein the ROM data is selected from the group consisting of flexion, extension and rotation, in combination with EMG data.

8. The method as specified in claim 1 wherein the ROM data is selected from the group consisting of flexion, extension and rotation, in combination with FCA data.

9. The method as specified in claim 1 wherein the FCA data is selected from the group consisting of lifting, pulling and pushing, in combination with ROM data, and spinal range of motion data.

10. The method as specified in claim 1 wherein the FCA data is selected from the group consisting of lifting, pushing and pushing, in combination with EMG data.

11. The method as specified in claim 1 wherein said EFA diagnostic data is acquired pre-surgical implantation of the hardware.

12. The method as specified in claim 1 wherein said EPA diagnostic data is acquired post-surgical implantation of the hardware.

13. The method as specified in claim 1 wherein said EFA data is acquired prior to pending hardware removal.

14. The method as specified in claim 1 wherein said EFA diagnostic data is acquired from a test protocol that comprises the steps:
a) select an individual who is a candidate for either pre-surgical hardware implantation, post-surgical hardware implantation or pre-removal of surgically-implanted hardware,
b) determine if the hardware removal has organic or objective etiology,
c) prepare said EFA device for selected test protocol,
d) perform EMG test, if selected,
e) perform ROM test, if selected,
f) perform FCA test, if selected,
g) acquire test protocol results and diagnostic data, wherein the diagnosis data will disclose if muscle, ischemic or nerve damage is present in the area of the surgically-implanted hardware, and
h) use the diagnostic data to determine whether pre-implanted hardware should be implanted, or if post-implanted hardware should be removed.

15. The method as specified in claim 14 wherein the test protocol further comprises diagnostic data acquired from paraspinal muscles and muscles surrounding the paraspinal region to determine if said data correlates to the location of the surgically-implanted hardware.

16. A method for determining if surgically-implanted cervical, thoracic or lumbar hardware should be removed from an individual, wherein said method utilizes diagnostic data acquired from a test protocol performed by an electrodiagnostics functional assessment (EFA) device, wherein said diagnostic data is selected from the group consisting of electromyography (EMG), range-of-motion (ROM), functional capacity assessment (ECA), muscle data, myofascial data, structural data, nerve data or ischemic data, wherein said test protocol comprises the steps:
a) select an individual who is a candidate for either pre-surgical hardware implantation, post-surgical hardware implantation or pre-removal of surgically-implanted hardware,
b) determine if the hardware removal has organic or objective etiology,
c) prepare said EFA device for selected test protocol,
d) perform at least one rest and return to rest test protocol for EMG, if selected,
e) perform at least one rest and return to rest test for ROM, if selected,
f) perform at least one rest and return to rest test for FCA, if selected,
g) acquire test protocol results and diagnostic data, wherein the diagnosis data will disclose if muscle, ischemic or nerve damage is present in the area of the surgically-implanted hardware, and
h) use the diagnostic data to determine whether pre-implanted hardware should be implanted, or if post-implanted hardware should be removed.

17. The method as specified in claim 16 wherein the surgically implanted cervical or lumbar hardware is selected from the group consisting of interbody screws, plates, rods, rods with pedicle screws, translaminar screws or facet screws.

18. The method as specified in claim 16 wherein said EMG data is evaluated bilaterally with spinal range of motion data.

19. The method as specified in claim 16 wherein said EMG data is evaluated bilaterally with FCA data.

20. The method as specified in claim 16 wherein the ROM data is selected from the group consisting of flexion, extension and rotation, in combination with EMG data.

21. The method as specified in claim 16 wherein the ROM data is selected from the group consisting of flexion, extension and relation, in combination with FCA data.

22. The method as specified in claim 16 wherein the FCA data is selected from the group consisting of lifting, pulling and pushing, in combination with ROM data, and spinal range of motion data.

23. The method as specified in claim 16 wherein the FCA data is selected from the group consisting of lifting, pushing and pushing, in combination with EMG data.

24. The method as specified in claim 16 wherein said EFA diagnostic data is acquired pre-surgical implantation of the hardware.

25. The method as specified in claim 16 wherein said EFA diagnostic data is acquired post-surgical implantation of the hardware.

26. The method as specified in claim 16 wherein said EFA data is acquired prior to pending hardware removal.

27. The method as specified in claim 16 wherein the test protocol further comprises diagnostic data acquired from paraspinal muscles and muscles surrounding the paraspinal region to determine if said data correlates to the location of the surgically-implanted hardware.

28. A method for determining if surgically-implanted cervical, thoracic or lumbar hardware should be removed from an individual, wherein said method utilizes diagnostic data acquired from a test protocol performed by an electrodiagnostic functional assessment (EFA) device to determine whether the hardware should be removed or remain implanted, wherein said diagnostic data is comprised of electromyography (EMG), range-of-motion ((ROM), or functional capacity assessment (ECA), wherein said EFA monitors muscles, comprising paraspinal muscles, cervical muscles, upper extremity muscles, lower extremity muscles, gluteal muscles, hip muscles or sacro iliac muscles, wherein said EFA monitors medical data comprising myofascial data, structural data, nerve data or ischemic data, wherein said test protocol comprises the following steps:
a) select an individual who is a candidate for either pre-surgical hardware implantation, post-surgical hardware implantation or pre-removal of surgically-implanted hardware,
b) determine if the hardware removal has organic or objective etiology,
c) prepare said EFA device for selected test protocol,
d) perform at least one rest and return to rest test protocol for EMG, if selected,
e) perform at least one rest and return to rest test for ROM, if selected,
f) perform at least one rest and return to rest test for FCA, if selected,
g) acquire test protocol results and diagnostic data, wherein the diagnosis data will disclose if muscle, ischemic or nerve damage is present in the area of the surgically-implanted hardware, and
h) use the diagnostic data to decide whether pre-implanted hardware should be implanted, or if post-implanted hardware should be removed.

29. The method as specified in claim 28 wherein the test protocol further comprises diagnostic data acquired from paraspinal muscles and muscles surrounding the paraspinal region to determine if said data correlates to the location of the surgically-implanted hardware.
